(19) United States
(12) Patent Application Publication
(10) Pub. No.: US 2026/0216537 A1
(43) Pub. Date: Jul. 30, 2026

(54) ULTRASOUND FAT CAVITATION EQUIPMENT AND METHOD OF USING THE SAME

(71) Applicant: LEADERMED GROUP US CORPORATION, Cranbury, NJ (US)

(72) Inventor: Qiao, Joanne JIANG CHEN, Pennington, NJ (US)

(73) Assignee: LEADERMED GROUP US CORPORATION, Cranbury, NJ (US)

(21) Appl. No.: 19/144,659

(22) PCT Filed: Dec. 6, 2023
(86) PCT No.: PCT/US2023/082621

§ 371 Date: Jun. 30, 2025

Related U.S. Application Data
(60) Provisional application No. 63/436,863, filed on Jan. 3, 2023.

(51) Int. Cl. A61N 7/00

(52) U.S. Cl. CPC A61N 7/00; A61N 2007/0008; A61N 2007/0039

(57) ABSTRACT

The disclosure includes an ultrasound fat cavitation equipment and a method of using the same. The equipment comprises a movable transducer configured to generate ultrasound waves to cavitate fat cells and a first handpiece; the movable transducer can move vertically inside the first handpiece, in order to change the distance between the movable transducer and the region of interest.

### BACKGROUND

In the field of aesthetic medicine, the most promising techniques for noninvasive body sculpting focus on ultrasound-induced fat reduction. These fat reduction ultrasound devices offer a practical way to reduce subcutaneous fat pads without significant adverse reactions.

Ultrasonic or ultrasound cavitation involves the use of ultrasound technology to break down fat cells located beneath the skin. This non-surgical approach is effective in reducing cellulite and localized fat deposits. During this procedure, ultrasonic vibrations are applied to exert energy on fat cells.

Various devices employ different methods. Some utilize high-frequency ultrasound waves to eliminate fat cells by generating heat or raising temperatures, while others use lower-frequency waves to mechanically disrupt the membranes of fat cells. Despite generally positive clinical outcomes, challenges such as non-responders, diminishing effects over time, inconsistent fat reduction results, and occasional side effects are common. Hence, there is a growing need for more effective non-invasive fat reduction equipment.

### SUMMARY OF THE INVENTION

In the first aspect, the disclosure pertains to an ultrasound fat cavitation equipment that employs low-frequency, non-focused ultrasound waves. This equipment is designed for the purpose of cavitating adipose tissue and treating adipose within a specific area of interest. It comprises a movable transducer responsible for generating ultrasound waves to induce cavitation in fat cells. Additionally, there is a first handpiece that houses the movable transducer, allowing it to move vertically inside the first handpiece. This movement capability enables the adjustment of the distance between the movable transducer and the targeted region of interest as needed.

In certain embodiments, the ultrasound fat cavitation equipment further comprises one or more rods located within the first handpiece. The movable transducer is designed to slide along these rod(s) through either a mechanical switch, which is manipulated on the surface of the first handpiece, or through electronic control. This sliding mechanism allows for the convenient adjustment of the distance between the movable transducer and the region of interest, providing flexibility in targeting specific areas during treatment.

In some embodiments, the movable transducer is composed of:
A first transducer designed to generate ultrasound waves with a lower frequency, falling within the range of 25-50 kHz. More specifically, it can be set to frequencies such as 25-45 kHz, 30-45 kHz, 30-40 kHz, or even more preferably, 35 kHz or 40 kHz.
Additionally, there is a second transducer integrated into the device, which is configured to produce ultrasound waves with a higher frequency, typically within the range of 50-90 kHz. This frequency range can be further narrowed down to options like 55-90 kHz, 55-85 kHz, 55-75 kHz, or 55-70 kHz, with 60 kHz being a preferred frequency setting.

These transducers with variable frequency capabilities offer versatility in addressing different treatment needs and scenarios.

In some embodiments, the ultrasound fat cavitation equipment further comprise one or more pressure sensors situated on the outer surface at the top of the first handpiece. These pressure sensors are responsible for measuring and providing information about the pressure applied during the procedure, ensuring accurate monitoring and control for enhanced precision and safety.

In some embodiments, the ultrasound fat cavitation equipment also incorporate one or more temperature sensors positioned on the outer surface at the top of the first handpiece. These temperature sensors are designed to monitor and maintain the treatment temperature within a specified and safe range, contributing to the effectiveness and safety of the procedure.

In some embodiments, the skin-contacting end of the first handpiece's surface is textured with specific patterns, chosen from closed designs like ring and oval patterns and open designs like wavy, parallel, radiant, ring with openings, and oval with openings patterns, or mixed design with open and closed designs. These patterns serve various purposes, potentially enhancing energy distribution, reducing hotspots, improving contact, adding aesthetic appeal, facilitating ease of handling, and minimizing acoustic reflection during the ultrasound fat reduction procedure.

In certain embodiments, the ultrasound fat cavitation equipment is equipped with a second handpiece, specifically designed for producing ultrasound waves used in ultrasound imaging. This configuration results in the ultrasound fat cavitation equipment having two distinct handpieces: the first handpiece dedicated to ultrasound cavitation and the second handpiece specialized for ultrasound imaging. These handpieces are integrated into the equipment as part of separate modules, with the first handpiece being part of the ultrasound cavitation module and the second handpiece belonging to the ultrasound imaging module. This design allows for the combination of ultrasound cavitation for fat reduction and ultrasound imaging capabilities within a single machine featuring two handpieces. This setup offers the advantage of convenience, enabling operators to seamlessly switch between cavitation and imaging functions using the same device. The second handpiece is equipped with a probe responsible for emitting and receiving ultrasound waves, facilitating the capture of detailed internal structure images.

In the second aspect, the disclosure also includes a method of using the ultrasound fat cavitation equipment of the first aspect.

In some embodiments, the method of using the ultrasound fat cavitation equipment comprises using coupling medium to fill the gap between a treatment head of the first handpiece and the region of interest; wherein said coupling medium is selected from composition A, and/or composition B, and/or composition C, and/or composition D;
the composition A includes water, mineral oil, cetearyl alcohol, PEG-8, glycerol stearate, glycerin, PEG-100 stearate, cyclopentamethylene siloxane, cyclohexane siloxane, carbomer, triethanolamine, allantoin, cetearyl glucoside, phenoxyethanol, and methyl hydroxybenzoate;
the composition B includes water, propylene glycol, glycerin, carbomer, triethanolamine, p-hydroxyacetophenone, allantoin, dipotassium glycyrrhizinate, ribonucleic acid, and 1,2-pentanediol;
the composition C includes mineral oil, vitis vinifera seed oil, helianthus annus seed oil, citrus aurantium dulcis peel oil, tocopherol acetate, and hibiscus abelmoschus seed extract;
the composition D includes mRNA, and/or microRNA, and/or antisense RNA for collagen production.

During the ultrasound fat reduction treatment, the coupling medium gets absorbed into the skin and deliver many useful ingredients to tighten the skin. During treatment and the days after treatment, these ingredients will continue increase collagen production in skin cells at the treatment locations. In some embodiments, the ingredient includes mRNA, and/or microRNA, and/or antisense RNA for collagen production.

In some embodiments, the composition A further includes xanthan gum, dipotassium glycyrrhizinate, essence, ethylhexylglycerol, EDTA disodium, and glucose.

In some embodiments, the composition B further includes hydroxyethylcellulose, octyl hydroxamic acid, sodium hyaluronate, punica granatum peel extract, nonylphenol polyether-15, butanediol, essence, nonylphenol polyether-10, camellia sinensis extract, glycerol octanoate, ellagic acid, galla rhois extract, 1,2-hexanediol, glutathione, polysorbate-60, disodium hydrogen phosphate, acetylhexapeptide-8, and sodium dihydrogen phosphate.

This feature of the movable transducer can provide several benefits:
1. Treatment Depth Control: The vertical movement of the transducer allows for control over the treatment depth. By adjusting the position of the transducer, the ultrasound fat cavitation equipment can target different layers of fat or tissue, depending on the specific treatment objectives and the depth at which the cavitation effect is desired.
2. Customizable Treatment Areas: The ability to move the transducer up and down enables the treatment of different areas or contours of the body. It allows for flexibility in adapting to various body shapes and sizes, ensuring that the ultrasound energy is effectively delivered to the desired treatment areas.
3. Enhanced Treatment Precision: Precise vertical positioning of the transducer can improve treatment accuracy. It allows for better alignment with the targeted treatment area, minimizing the risk of unnecessary exposure to surrounding tissues and optimizing the effectiveness of the ultrasound waves.

The transducer, no matter is the movable transducer, the unmovable transducer, the first transducer or the second transducer, is a crucial component within the first handpiece that generates the ultrasound waves. The design may include a single transducer or an array of transducers depending on the specific machine. The shape and positioning of the transducer(s) within the first handpiece can affect the efficiency and targeting capabilities of the ultrasound energy.

The transducer converts electrical energy into mechanical vibrations that produce the ultrasound waves used for the cavitation process. As it consists of one or more piezoelectric elements. These elements, typically made of ceramic or crystal materials, possess the ability to deform when subjected to an electric field. This deformation generates the mechanical vibrations necessary for ultrasound wave generation.

### BRIEF DESCRIPTION OF THE DRAWINGS

FIG. 1 shows the surface of the skin contacting end of the first handpiece with a mixed design (DESIGN 1), consisting of ovals and arcs, when viewed from the top.

FIG. 2 shows the surface of the skin contacting end of the first handpiece with a mixed design (DESIGN 2), consisting of ring and arcs, when viewed from the top.

FIG. 3 shows the surface of the skin contacting end of the first handpiece with a rings design (DESIGN 3) when viewed from the top.

FIG. 4 shows the surface of the skin contacting end of the first handpiece with an ovals design (DESIGN 4) when viewed from the top.

FIG. 5 shows the surface of the skin contacting end of the first handpiece with a wavy design (DESIGN 5) when viewed from the top.

FIG. 6 shows the surface of the skin contacting end of the first handpiece with a parallel design (DESIGN 6), consisting of some straight parallel lines when viewed from the top.

FIG. 7 shows the surface of the skin contacting end of the first handpiece with a parallel design (DESIGN 7), consisting of some arcs parallel lines and a straight line in the middle, when viewed from the top.

FIG. 8 shows the surface of the skin contacting end of the first handpiece with a radiant design (DESIGN 8), consisting of some straight lines, when viewed from the top.

FIG. 9 shows the surface of the skin contacting end of the first handpiece with a radiant design (DESIGN 9), consisting of some wavy lines, when viewed from the top.

FIG. 10 shows the surface of the skin contacting end of the first handpiece with an open design (DESIGN 10), consisting of some rings with openings, when viewed from the top.

FIG. 11 shows the surface of the skin contacting end of the first handpiece with an open design (DESIGN 11), consisting of some ovals with openings, when viewed from the top.

FIG. 12 shows the surface of the skin contacting end of the first handpiece with a raised rings design (DESIGN 3) when viewed from the side.

FIG. 13 shows the surface of the skin contacting end of the first handpiece with a raised mixed design (DESIGN 1), consisting of ovals and arcs, when viewed from the side.

FIG. 14 shows the surface of the skin contacting end of the first handpiece with a raised radiant design (DESIGN 8), consisting of some straight lines, when viewed from the side.

FIG. 15 shows the surface of the skin contacting end of the first handpiece with a concave rings design (DESIGN 3) when viewed from the side.

FIG. 16 shows the surface of the skin contacting end of the first handpiece with a concave mixed design (DESIGN 1), consisting of ovals and arcs, when viewed from the side.

FIG. 17 shows the surface of the skin contacting end of the first handpiece with a concave radiant design (DESIGN 8), consisting of ovals and arcs, when viewed from the side.

FIG. 18 shows the surface of the skin contacting end of the first handpiece with a raised wavy design (DESIGN 5), consisting of some straight lines, when viewed from the side.

FIG. 19 shows the surface of the skin contacting end of the first handpiece with a concave wavy design (DESIGN 5), consisting of some straight lines, when viewed from the side.

FIG. 20 shows schematic diagram of the handpiece comprising a movable transducer in some embodiments.

FIG. 21 shows schematic diagram of the first handpiece comprising a first transducer and a second transducer in some embodiments.

FIG. 22 shows schematic diagram of the first handpiece comprising sensors in some embodiments with sensors flushing with the outer surface of the treatment head of the first handpiece.

FIG. 23 shows schematic diagram of the first handpiece comprising sensors in some embodiments with membrane structure of sensors.

### DETAILED DESCRIPTION OF THE INVENTION

The technical solution of the present invention will be further described below with reference to the accompanying drawings and specific embodiments.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

The disclosure includes an ultrasound fat cavitation equipment using non-focused ultrasound low frequency waves and method of using the same. The ultrasound fat cavitation equipment for cavitating adipose tissue and for treating adipose in a region of interest, comprises a movable transducer configured to generate ultrasound waves to break fat cells and a first handpiece; the movable transducer can move up and down inside the first handpiece, in order to change the distance between the movable transducer and the region of interest.

In some embodiments, the ultrasound fat cavitation equipment further comprises one or more rods inside of the first handpiece; the movable transducer is configured to slide along the rod(s) by a mechanical switch pushing control on the surface of the first handpiece or by electronical control to change the distance between the movable transducer and the region of interest.

In some embodiments, the distance between the movable transducer and the region of interest is arranged from 0.5-5.0 cm.

In some embodiments, the movable transducer comprises:
a first transducer configured to generate ultrasound waves with lower frequency, in the range of 25-50 kHz, preferably is 25-45 kHz, 30-45 kHz, 30-40 kHz, more preferably is 35 kHz or 40 kHz; and/or
a second transducer configured to generate ultrasound waves with higher frequency, in the range of 50-90 kHz, preferably is 55-90 kHz, 55-70 kHz, more preferably is 60 kHz.

In some embodiments, the number of the first transducer is one, and the number of the second transducers is more than one, preferably is 2-10 or 2-6 or 2-3, and the second transducers are configured to surround the first transducer.

In some embodiments, the ultrasound fat cavitation equipment further comprises an unmovable transducer configured to generate ultrasound waves with lower frequency, in the range of 25-50 kHz, preferably is 25-45 kHz, 30-50 kHz, 30-45 kHz, or 30-40 kHz, more preferably is 35 kHz or 40 kHz; the movable transducer is configured to generate ultrasound waves with higher frequency, in the range of 50-90 kHz, preferably is 55-90 kHz, 55-85 kHz, or 55-70 kHz, more preferably is 60 kHz.

In some embodiments, the ultrasound fat cavitation equipment further comprises an unmovable transducer configured to generate ultrasound waves with higher frequency, in the range of 50-90 kHz, preferably is 55-85 kHz, 55-70 kHz, more preferably is 60 kHz; the movable transducer is configured to generate ultrasound waves with lower frequency, in the range of 25-45 kHz, preferably is 30-45 kHz, 30-40 kHz, more preferably is 35 or 40 kHz.

In some embodiments, the ultrasound fat cavitation equipment further comprises one or more pressure sensors, which is/are on the outer surface of the top of the first handpiece.

In some embodiments, the pressure sensor(s) is/are wired sensor(s) or wireless sensor(s).

In some embodiments, the ultrasound fat cavitation equipment further comprises one or more temperature sensors, which is/are on the outer surface of the top of the first handpiece.

In some embodiments, the temperature sensor(s) is/are wired sensor(s) or wireless sensor(s).

In some embodiments, the ultrasound fat cavitation equipment further comprises a cooling system, an acoustic focusing mechanism, and a housing. The cooling system includes heat sinks and/or cooling fans to dissipate excess heat generated by a transducer (it can be the movable transducer, the unmovable transducer, the first transducer and/or the second transducer). The heat sinks and the cooling fans are inside of the first handpiece.

The acoustic focusing mechanism helps concentrate the ultrasound waves to a specific treatment area, improving the precision and effectiveness of the cavitation process. The acoustic focusing mechanism is also inside of the first handpiece and is between the transducer and the treatment head of the first handpiece.

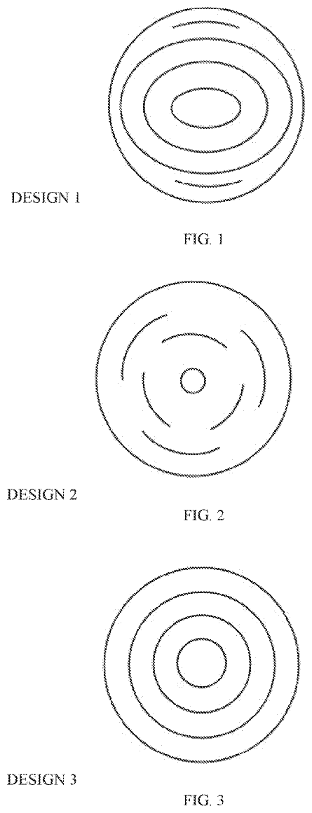

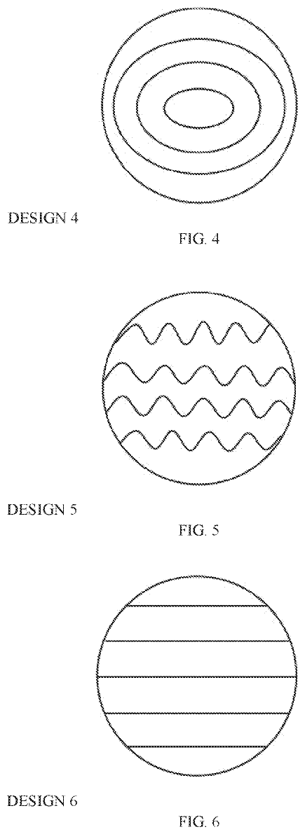

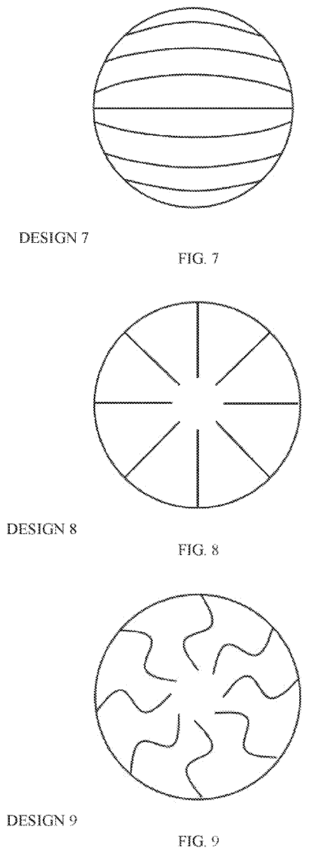

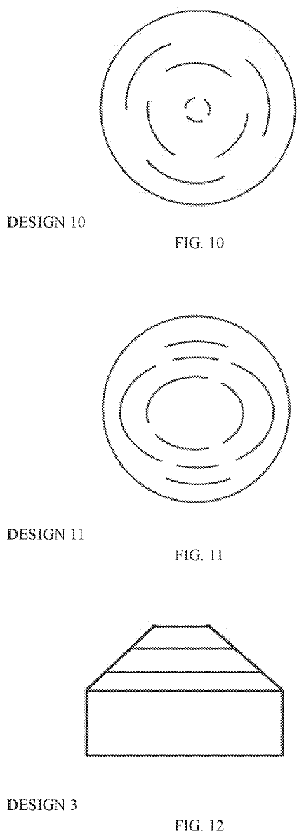

In some embodiments, the surface of the skin contacting end of the first handpiece has textured with certain patterns so that it will potentially have enhanced energy distribution, reduced hotspots, improved contact, aesthetic appeal, ease of handling, and reduced acoustic reflection. The certain patterns are selected from closed design, open design and mixed design (e.g. FIGS. 1 and 2) with both closed design and open design. The closed design includes one or more of ring design (e.g. FIG. 3) and oval design (e.g. FIG. 4). The open design includes one or more of wavy design (e.g. FIG. 5), parallel design (e.g. FIGS. 6 and 7), radiant design (e.g. FIGS. 8 and 9), ring with openings design (e.g. FIG. 10), and oval with openings design (e.g. FIG. 11).

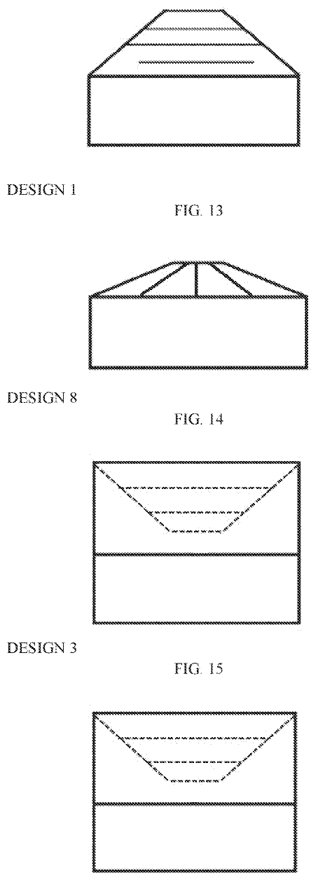

In some embodiments, the surface of the skin contacting end is raised, preferably the center of the surface is raised, more preferably the center of the surface is gradually raised (e.g. FIG. 12 to 14).

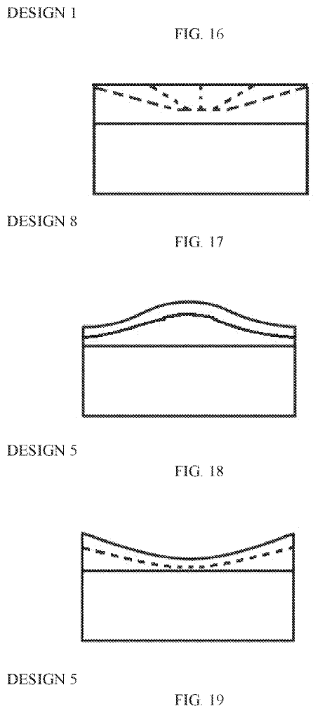

In some embodiments, the surface of the skin contacting end is concave, preferably with a concavity in the center of the surface, more preferably with a gradual concavity in the center of the surface (e.g. FIG. 15 to 17).

In some embodiments, the surface of the skin contacting end is height variant, such as the center wavy line of the wavy design is raised higher than the side wavy lines of it (e.g. FIG. 18), or the center wavy line of the wavy design is concave deeper than the side wavy lines of it (e.g. FIG. 19).

FIG. 1 to 11 show the top view of DESIGNS 1 to 11, respectively. These DESIGNS are raised or concave when viewed from the side. For example, FIG. 12 to 14 and FIG. 18 show that DESIGN 3, DESIGN 1, DESIGN 8, and DESIGN 5 are raised, respectively. FIG. 15 to 17 and FIG. 19 show that DESIGN 3, DESIGN 1, DESIGN 8, and DESIGN 5 are concave, respectively.

In some embodiments, the surface of the skin contacting end is height variant, such as the center parallel line of the parallel design is raised higher than the side parallel lines of it, or the center parallel line of the parallel design is raised lower than the side parallel lines of it.

In some embodiments, the ultrasound fat cavitation equipment further comprises a ear protection element, such as earplugs, earmuffs, noise-canceling earbuds, noise-canceling headphones, and custom-made ear protection.

In some embodiments, the noise-canceling earbuds and/or the noise-canceling headphones are selected from Bose QuietComfort Earbuds, Sony WF-1000XM4, Apple Airpods pro and Jabra Elite 85t.

In some embodiments, the ultrasound fat cavitation equipment further comprises a second handpiece, which can produce ultrasound waves using for ultrasound imaging. The ultrasound fat cavitation equipment further comprises an ultrasound cavitation module and an ultrasound imaging module. The first handpiece is belonged to the ultrasound cavitation module, and the second handpiece is belonged to the ultrasound imaging module.

The ultrasound cavitation module involves the application of low-frequency ultrasound waves to the targeted area of the body. These ultrasound waves create microbubbles in the fat tissue, causing them to expand and contract rapidly. The continuous expansion and contraction of these bubbles generate pressure changes within the fat cells, leading to their breakdown. The broken-down fat cells release their contents, which are then metabolized and eliminated by the body's natural processes.

The ultrasound imaging module is a component that uses ultrasound waves to create images of structures within the body. It works by emitting high-frequency sound waves that bounce off internal structures and return as echoes. These echoes are then used to create real-time images of organs, tissues, and other structures. The ultrasound imaging module has an ultrasound imaging transducer inside of the second handpiece. The ultrasound imaging transducer is capable of emitting and receiving ultrasound waves for real-time imaging.

The ultrasound fat cavitation equipment further comprises a control interface and an imaging display. The control interface would allow the operator to control both the cavitation and imaging functions, and to select which function to use and adjust the corresponding parameters. The corresponding parameters could include settings for adjusting energy levels, treatment duration, and imaging parameters.

The imaging display is a screen to display the real-time ultrasound images captured by the ultrasound imaging module. This display would aid the operator in visualizing the treatment area during the procedure, such as fat deposits, muscles, and other tissues. With the ultrasound imaging module's guidance, the operator can identify the specific fat deposits to be targeted for the cavitation procedure. The operator can adjust the position and orientation of the treatment device based on the imaging feedback to ensure precise targeting. with the ultrasound imaging module, the operator can monitor the effects of the cavitation in real-time, ensuring that the fat cells are being effectively broken down while minimizing impact on surrounding tissues. The operator can use post-treatment imaging to assess the immediate effects of the procedure and ensure uniform treatment coverage.

### Workflow: The Operator's Workflow With a Dual-Function Machine Would Involve the Following Steps:

1. Imaging Phase:

The operator selects the imaging function on the control interface.

The imaging handpiece (the second handpiece) is used to scan the treatment area, capturing real-time ultrasound images.
2. Targeting and Planning:

Based on the imaging feedback, the operator identifies the areas for fat reduction and plans the treatment approach.
3. Cavitation Phase:

The operator switches to the cavitation function on the control interface.

The cavitation handpiece (the first handpiece) is used to apply the low-frequency ultrasound waves to the targeted fat deposits.
4. Real-time Monitoring:

Throughout the cavitation procedure, the operator can switch between two modules and periodically.

The operator can monitor the treatment progress and effects on fat cells in real-time.
5. Evaluation and Documentation:

After completing the cavitation procedure, the operator can use the imaging handpiece to assess the immediate effects and ensure uniform treatment coverage.

The captured images can also be used for documentation and patient records.

Skin Type: The patient's skin type classification (e.g., Fitzpatrick scale), which can affect how the ultrasound energy is absorbed and transmitted through the skin.

Target Area: The specific area of the body being targeted for the ultrasound cavitation treatment, as different body parts may have varying tissue properties and treatment requirements.

Medical History: Relevant medical history of the patient, including any pre-existing conditions, allergies, or previous treatments that may impact the suitability or effectiveness of the ultrasound cavitation treatment.

Medications: Information about any medications or supplements the patient is currently taking, as certain medications can interact with the treatment or affect the healing process.

Sensitivity/Pain Tolerance: The patient's sensitivity or pain tolerance level, which can influence the intensity or duration of the treatment.

Lifestyle Factors: Factors such as physical activity level, smoking status, and dietary habits that may affect treatment outcomes and the overall success of the ultrasound cavitation treatment.

The data (pressure data) from a pressure sensor can affect the transmission of ultrasound waves and their interaction with the target tissue. By monitoring and incorporating the pressure as an input feature, the machine learning module can account for its impact on treatment outcomes. This information can help optimize the treatment parameters to ensure an appropriate and consistent level of pressure for effective and safe results.

Monitoring the temperature during the treatment by the temperature sensor can provide valuable insights into the thermal effects of ultrasound cavitation. It can help assess whether the temperature remains within a safe and effective range to avoid potential tissue damage. By including temperature measurements as input data, the machine learning module can learn the relationship between temperature changes and treatment outcomes. This knowledge can guide the optimization of treatment parameters to achieve the desired therapeutic effects while maintaining a suitable temperature range, and also can guide the control and data processing module to control the cooling system to lower the temperature.

In some embodiments, the method of using the ultrasound fat cavitation equipment comprises using coupling medium to fill the gap between a treatment head of the first handpiece and the region of interest; wherein said coupling medium is selected from composition A, and/or composition B, and/or composition C, and/or composition D;
the composition A includes water, mineral oil, cetearyl alcohol, PEG-8, glycerol stearate, glycerin, PEG-100 stearate, cyclopentamethylene siloxane, cyclohexane siloxane, carbomer, triethanolamine, allantoin, cetearyl glucoside, phenoxyethanol, and methyl hydroxybenzoate;
the composition B includes water, propylene glycol, glycerin, carbomer, triethanolamine, p-hydroxyacetophenone, allantoin, dipotassium glycyrrhizinate, ribonucleic acid, and 1,2-pentanediol;
the composition C includes mineral oil, vitis vinifera seed oil, helianthus annus seed oil, citrus aurantium dulcis peel oil, tocopherol acetate, and hibiscus abelmoschus seed extract;
the composition D includes mRNA, and/or microRNA, and/or antisense RNA for collagen production.

During the ultrasound fat reduction treatment, the coupling medium gets absorbed into the skin and deliver many useful ingredients to tighten the skin. During treatment and the days after treatment, these ingredients will continue increase collagen production in skin cells at the treatment locations.

In some embodiments, the ingredient includes mRNA, and/or microRNA, and/or antisense RNA for collagen production.

### First Embodiment

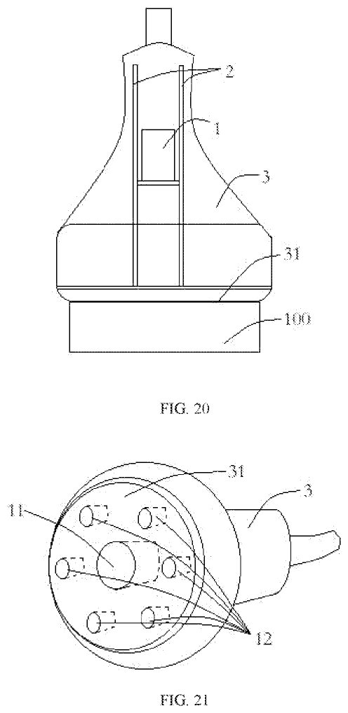

In this embodiment (FIG. 20), the ultrasound fat cavitation equipment for cavitating adipose tissue and for treating adipose in a region of interest 100 comprises a movable transducer 1, two rods 2, and a first handpiece 3. The movable transducer 1 is configured to slide along the rods 2 by a mechanical switch pushing control on the surface of the first handpiece to change the distance between the movable transducer 1 and the region of interest 100. The movable transducer 1 and the two rods 2 are inside the first handpiece 3. The two rods 2 are vertical to the top of the first handpiece. The top of the first handpiece also known as the treatment head 31 of the first handpiece.

### Second Embodiment

In this embodiment (FIG. 21), the ultrasound fat cavitation equipment for cavitating adipose tissue and for treating adipose in a region of interest comprises a movable transducer, two rods, and a first handpiece 3. The movable transducer is configured to slide along the rods by electronical control to change the distance between the movable transducer and the region of interest.

The movable transducer comprises a first transducer 11 and a second transducer 12. The first transducer 11 configured to generate ultrasound wave with lower frequency, in the range of 25-45 kHz, preferably is 30-40 kHz, more preferably is 35 kHz. The second transducer 12 configured to generate ultrasound wave with higher frequency, in the range of 55-85 kHz, preferably is 55-75 kHz, more preferably is 60 kHz. The number of the first transducer 11 is one, and the number of the second transducers 12 is 6. The second transducers 12 are in a circle, and the first transducer 11 is in the center of the circle.

### Third Embodiment

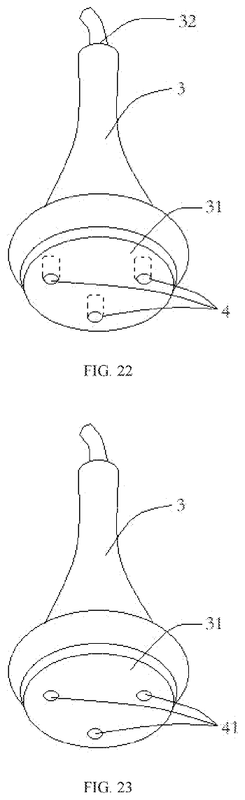

In this embodiment (FIG. 22), the ultrasound fat cavitation equipment for cavitating adipose tissue and for treating adipose in a region of interest comprises a movable transducer, two rods, some sensors and a first handpiece 3. The movable transducer is configured to slide along the rods by electronical control to change the distance between the movable transducer and the region of interest. The sensors are wired pressure sensors 4 (they can be wired temperature sensors, or can be wired pressure sensors and wired temperature sensors, in some embodiments). The detection interface of the wired pressure sensors 4 are flush with the outer surface of the treatment head 31 of the first handpiece. The wire of the wired pressure sensors 4 gets through the tail 32 of the first handpiece to connect to the control and data processing module of the ultrasound fat cavitation equipment.

### Forth Embodiment

In this embodiment (FIG. 23), the ultrasound fat cavitation equipment for cavitating adipose tissue and for treating adipose in a region of interest comprises a movable transducer, two rods, some sensors and a first handpiece 3. The movable transducer is configured to slide along the rods by electronical control to change the distance between the movable transducer and the region of interest.

The sensors are wireless pressure sensors 41 (they can be wired temperature sensors or can be wired pressure sensors and wired temperature sensors, in some embodiments). The wireless pressure sensors 41 are membrane structures that are applied to the outer surface of the treatment head 31 of the first handpiece. The wireless pressure sensors 41 use blue tooth to exchange data with the control and data processing module of the ultrasound fat cavitation equipment.

### Fifth Embodiment

In this embodiment, the sensors are wireless pressure sensors (they can be wired temperature sensors, or can be wired pressure sensors and wired temperature sensors, in some embodiments). The wireless pressure sensors are membrane structures that are applied to the outer surface of the treatment head of the first handpiece. The wireless pressure sensors use blue tooth to exchange data with the control and data processing module of the ultrasound fat cavitation equipment. The rest is the same as in the first embodiment.

### Sixth Embodiment

In this embodiment, the method of using the ultrasound fat cavitation equipment disclosure herein comprises using coupling medium to fill the gap between a treatment head of the first handpiece and the region of interest.

The coupling medium comprises composition A. The composition A includes water, mineral oil, cetearyl alcohol, PEG-8, glycerol stearate, glycerin, PEG-100 stearate, cyclopentamethylene siloxane, cyclohexane siloxane, carbomer, triethanolamine, allantoin, cetearyl glucoside, phenoxyethanol and methyl hydroxybenzoate. The composition A further includes xanthan gum, dipotassium glycyrrhizinate, essence, ethylhexylglycerol, EDTA disodium and glucose as trace components.

### Seventh Embodiment

In this embodiment, the coupling medium further comprises composition B. The composition B includes water, propylene glycol, glycerin, carbomer, triethanolamine, p-hydroxyacetophenone, allantoin, dipotassium glycyrrhizinate, ribonucleic acid and 1,2-pentanediol. The composition B further includes hydroxyethylcellulose, octyl hydroxamic acid, sodium hyaluronate, punica granatum peel extract, nonylphenol polyether-15, butanediol, essence, nonylphenol polyether-10, camellia sinensis extract, glycerol octanoate, ellagic acid, galla rhois extract, 1,2-hexanediol, glutathione, polysorbate-60, disodium hydrogen phosphate, acetylhexapeptide-8 and sodium dihydrogen phosphate as trace components. The rest is the same as in the sixth embodiment.

### Eighth Embodiment

In this embodiment, the coupling medium further comprises composition C. The composition C includes mineral oil, vitis vinifera seed oil, helianthus annus seed oil, citrus aurantium dulcis peel oil, tocopherol acetate and hibiscus abelmoschus seed extract. The rest is the same as in the sixth embodiment.

While only certain features and embodiments of the invention have been illustrated and described herein, many modifications and changes will occur to those skilled in the art. It is, therefore, to be understood that the appended claims are intended to cover all such modifications and changes as fall within the scope of the invention.

1. An ultrasound fat cavitation equipment for cavitating adipose tissue and for treating adipose in a region of interest, wherein the ultrasound fat cavitation equipment comprises a movable transducer configured to generate ultrasound waves to cavitate fat cells and a first handpiece, and the movable transducer can move vertically inside the first handpiece, in order to change the distance between the movable transducer and the region of interest.

2. The ultrasound fat cavitation equipment of claim 1, further comprising one or more rods inside the first handpiece, wherein the movable transducer is configured to slide along these rod(s) through either a mechanical, which is manipulated on the surface of the first handpiece on the surface of the first handpiece, or through electronic control to change the distance between the movable transducer and the region of interest.

3. The ultrasound fat cavitation equipment of claim 1, wherein the movable transducer comprises:
a first transducer configured to generate ultrasound waves with a lower frequency, in the range of 25-50 kHz, preferably is 25-45 kHz, 30-45 kHz, or 30-40 kHz, more preferably is 35 kHz or 40 kHz; and/or
a second transducer configured to generate ultrasound waves with a higher frequency, in the range of 50-90 kHz, preferably is 55-90 kHz, 55-85 kHz, 55-75 kHz, or 55-70 kHz, more preferably is 60 kHz.

4. The ultrasound fat cavitation equipment of claim 3, wherein the number of the first transducer is one, and the number of the second transducers is more than one, preferably is 2-10 or 2-6 or 2-3, and the second transducers are configured to surround the first transducer.

5. The ultrasound fat cavitation equipment of claim 1, further comprising an unmovable transducer configured to generate ultrasound waves with lower frequency, in the range of 25-50 kHz, preferably is 25-45 kHz, 30-50 kHz, 30-45 kHz, or 30-40 kHz, more preferably is 35 kHz or 40 kHz: wherein the movable transducer is configured to generate ultrasound waves with higher frequency, in the range of 50-90 kHz, preferably is 55-90 kHz, 55-85 kHz, or 55-70 kHz, more preferably is 60 kHz.

6. The ultrasound fat cavitation equipment of claim 1, further comprising an unmovable transducer configured to generate ultrasound waves with higher frequency, in the range of 50-90 kHz, preferably is 55-90 kHz, 55-85 kHz, or 55-70 kHz, more preferably is 60 kHz; wherein the movable transducer is configured to generate ultrasound waves with lower frequency, in the range of 25-45 kHz, 30-50 kHz, 30-45 kHz, or 30-40 kHz, more preferably is 35 kHz or 40 kHz.

7. The ultrasound fat cavitation equipment of claim 1, further comprising one or more sensors situated on the outer surface of the top of the first handpiece; the sensor(s) is/are selected from a pressure sensor, a temperature sensor.

8. The ultrasound fat cavitation equipment of claim 7, wherein the sensor(s) is/are wired sensor(s) or wireless sensor(s).

9. The ultrasound fat cavitation equipment of claim 1, wherein the skin-contacting end of the first handpiece's surface is textured with certain patterns, which are selected from closed design, open design and mixed design with both closed design and open design.

10. The ultrasound fat cavitation equipment of claim 9, wherein the surface of the skin contacting end is raised, preferably the center of the surface is raised, more preferably the center of the surface is gradually raised.

11. The ultrasound fat cavitation equipment of claim 9, wherein the surface of the skin contacting end is concave, preferably with a concavity in the center of the surface, more preferably with a gradual concavity in the center of the surface.

12. The ultrasound fat cavitation equipment of claim 1, further comprising a second handpiece, which can produce ultrasound waves using for ultrasound imaging.

13. A method of using the ultrasound fat cavitation equipment of claim 1 comprising: using coupling medium to fill the gap between a treatment head of the first handpiece and the region of interest; wherein said coupling medium is selected from composition A, and/or composition B, and/or composition C, and/or composition D; the composition A includes at least 1, 2, 3, 4, 5, 6, 7, 8, 9, 10, 11, 12, 13, 14 or all 15 of water, mineral oil, cetearyl alcohol, PEG-8, glycerol stearate, glycerin, PEG-100 stearate, cyclopentamethylene siloxane, cyclohexane siloxane, carbomer, triethanolamine, allantoin, cetearyl glucoside, phenoxyethanol, and methyl hydroxybenzoate; the composition B includes at least 1, 2, 3, 4, 5, 6, 7, 8, 9, or all 10 of water, propylene glycol, glycerin, carbomer, triethanolamine, p-hydroxyacetophenone, allantoin, dipotassium glycyrrhizinate, ribonucleic acid, and 1,2-pentanediol; the composition C includes at least 1, 2, 3, 4, 5, or all 6 of mineral oil, vitis vinifera seed oil, helianthus annus seed oil, citrus aurantium dulcis peel oil, tocopherol acetate, and hibiscus abelmoschus seed extract; the composition D includes mRNA, and/or microRNA, and/or antisense RNA for collagen production.

14. The method of claim 13, wherein the composition A further includes at least 1, 2, 3, 4, 5, or all 6 of xanthan gum, dipotassium glycyrrhizinate, essence, ethylhexylglycerol, EDTA disodium, and glucose.

15. The method of claim 13, wherein the composition B further includes at least 1, 2, 3, 4, 5, 6, 7, 8, 9, 10, 11, 12, 13, 14, 15, 16, 17, 18, or all 19 of hydroxyethylcellulose, octyl hydroxamic acid, sodium hyaluronate, punica granatum peel extract, nonylphenol polyether-15, butanediol, essence, nonylphenol polyether-10, camellia sinensis extract, glycerol octanoate, ellagic acid, galla rhois extract, 1,2-hexanediol, glutathione, polysorbate-60, disodium hydrogen phosphate, acetylhexapeptide-8, and sodium dihydrogen phosphate.
